(19) United States
(12) Patent Application Publication
(10) Pub. No.: US 2021/0206834 A1
(43) Pub. Date: Jul. 8, 2021

(54) COMPOSITIONS AND METHODS FOR TREATMENT OF OCULAR DISEASES

(71) Applicant: MOR RESEARCH APPLICATIONS LTD., Ramat Gan (IL)

(72) Inventors: Dov WEINBERGER, Tel Aviv (IL); Tami LIVNAT, Savyon (IL); Yael NISGAV, Ramat Hasharon (IL)

(21) Appl. No.: 17/154,864

(22) Filed: Jan. 21, 2021

Related U.S. Application Data
(63) Continuation-in-part of application No. 15/780,294, filed on May 31, 2018.
(60) Provisional application No. 62/262,394, filed on Dec. 3, 2015.

(51) Int. Cl. C07K 14/745; A61P 27/02; A61K 45/06

(52) U.S. Cl. CPC C07K 14/745; A61K 38/00; A61K 45/06; A61P 27/02

(57) ABSTRACT

Methods for treating and/or preventing choroidal neovascularization (CNV) or retinal leakage associated with CNV are disclosed, comprising the use of activated protein C (APC) and/or an APC variant. A disclosed method may be applied in the treatment or prevention of ocular diseases, disorders or conditions that are caused directly by CVN, feature development of CNV as a secondary stage or a complication thereof, and/or feature CNV as a synchronous or asynchronous sequela thereof. An exemplary disease treatable by a disclosed method is neovascular age-related macular degeneration (nAMD).

### TECHNICAL FIELD

The present disclosure relates to treatment and/or prevention of retinal leakage and choroidal neovascularization, more particularly, but not exclusively, to treatment and/or prevention of retinal leakage and choroidal neovascularization in ocular diseases.

### BACKGROUND

Choroidal neovascularization (CNV) is the creation of new blood vessels in the choroid layer of the eye, beneath the retina. Choroid is the area between the retina and the sclera (the white part of the eye) that supplies oxygen and nutrients to the eye. CNV occurs when new blood vessels start to grow in the choroid and break through Bruch's membrane (BrM), the barrier between the choroid and the retina that supports the retina, into the retina and disrupt it. After choriocapillaries initially penetrate BrM, invading vessels may regress or expand and result in CNV. During early and late CNV, the expanding vasculature usually spreads in at least one of two distinct patterns: in a layer between BrM and the retinal pigment epithelium (RPE, the black lining of the eye) (sub-RPE or Type 1 CNV), in a layer between the RPE and the photoreceptors (sub-retinal or Type 2 CNV). When the two patterns occur simultaneously, CNV is diagnosed as combined pattern or Type 3 CNV. CNV leaking in the retina, e.g., bleeding and exudation, accounts for some acute visual symptoms, most prominent of which is vision loss.

The location, growth pattern and type of CNV depend on the patient's age and the underlying disease with which it is associated. CNV is the leading cause of severe vision loss in various ocular diseases such as age-related macular degeneration (AMD) particularly neovascular age-related macular degeneration (nAMD), myopathy and angioid streaks.

Classic and occult CNV are distinguished, although they can appear in combined forms. Occult CNVs proliferate under the RPE. Classic CNVs which are less frequent than occult or mixed forms, can occur in exudative AMD, as well as secondary to other chorioretinal diseases. Classic CNV is diagnosed by angiography, wherein it is defined as a clearly visible and well-demarcated hyperfluorescence in the early phase angiography, caused by vascular proliferations between the RPE and the neuroretina, with increasing leakage in the late phase of the angiography. The initial symptoms of classic CNV are metamorphopsia, deterioration in visual acuity, and central visual field defects.

Ophthalmoscopic signs of CNV are grayish-white sub-retinal changes together with retinal edema, hard exudations, and subretinal and intraretinal hemorrhage. If the condition is not treated, progression with enlargement of the lesion and subsequent loss of photoreceptors will usually follow.

Most studies hypothesize that CNV primarily results from growth-factor effects or holes in BrM. However, a thorough computer simulation study (Shirinifard et al., 2012, PLoS Computational Biology, 8(5):1-32), based on three-dimensional simulations of multi-cell model of the normal and pathological maculae, hypothesized that the three growth patterns of CNV (Types 1-3) result from a combinations of one or more impairments: (1) RPE-RPE epithelial junctional adhesion; (2) adhesion of the RPE basement membrane complex to BrM (RPE-BrM adhesion); and (3) adhesion of the RPE to the photoreceptor outer segments (RPE-POS adhesion).

Key findings of the study were that when an endothelial tip cell penetrates BrM, one or more of the following may occur: (1) RPE with normal epithelial junctions, basal attachment to BrM and apical attachment to POS would resists CNV; (2) small holes in BrM do not, by themselves, initiate CNV; (3) RPE with normal epithelial junctions and normal apical RPE-POS adhesion, but weak adhesion to BrM (e.g. due to lipid accumulation in BrM) results in early sub-RPE CNV. Reduced adhesion between pigmented retinal cells and Bruch's membrane is the type of CNV typical of aging; (4) Normal adhesion of RBE to BrM but reduced apical RPE-POS or epithelial RPE-RPE adhesion (e.g., due to inflammation) results in Early sub-retinal CNV. Reduced adhesion between neighboring pigmented retinal cells is typical of inflammation due to severe infection and is a pattern of invasion seen in young adults; and (5) simultaneous reduction in RPE-RPE epithelial and RPE-BrM adhesions result in either sub-RPE or sub-retinal CNV which often progresses to combined pattern CNV.

There are many causes to CNV most of which is synchronous or non-synchronous occurrence of an ocular disease or disorder, or a systemic disease or disorder such as inflammation or an autoimmune disease or disorder. However, since adhesion is an essential mechanism in maintaining the retina's structure, it seems that defects in adhesion dominate CNV initiation and progression, subsequently followed by retinal leakage.

The two main current treatments for CNV are either eliminating the invading blood vessels with drugs, mostly anti-angiogenesis agents, injected into the eye (most often also damaging the retina and killing needed blood vessels as well), or laser occlusion of the blood vessels, which can cause damaging retinal scars. Yet neither treatment still addresses the underlying problems that cause the blood vessels to invade, therefore, relapses are common, and many patients still lose vision within a year or two.

Activated protein C (APC) is a plasma protease having two distinct functions. (1) anticoagulant properties mediated by proteolysis of coagulation factors Va and VIIIa; and (2) cytoprotective effects including anti-apoptotic effects, anti-inflammatory effects, neuroprotective effects, favorable alterations of gene expression and endothelial and epithelial barriers stabilization (Griffin et al., Blood, 2015, 125(19):2898-2907: Vetrano et al., 2011, PNAS, 108(49): 1983019835).

A recombinant APC (drotrecogin alfa) was approved for adult severe sepsis treatment; however, therapy was complicated by bleeding events that were considered drug-related side effects. A modified APC, 3K3A-APC, was designed to possess significantly reduced anticoagulant activity (<10%) while maintaining full cytoprotective properties, and thus diminishing the risk of bleeding (Mosnier et al., 2004, Blood, 104:1740-4).

### SUMMARY

When disruption of the homeostasis between the retinal pigment epithelium (RPE) and Bruch's membrane occurs, a vicious circle may lead to choroidal neo-angiogenesis also known as choroidal neovascularization (CNV). It is appreciated now that cell adhesion is one of the keys to keeping blood vessels out of the retina and that a combination of defects in at least one types of adhesion, namely, RPE-RPE epithelial junctional adhesion; adhesion of the RPE basement membrane complex to Bruch's membrane; or adhesion of the RPE to the photoreceptor outer segments, is sufficient to determine the probability, pattern, and rate of progression of CNV.

The present inventors have successfully addressed the yet unmet need for therapies which restore normal adhesion in the eye thereby providing means to treat CNV and inhibit or prevent deleterious choroidal neo-vascular leakage in the retina. These therapies comprise the use of APC.

In one aspect, the present disclosure relates to a method therapy of choroidal neovascularization (CNV) or retinal leakage associated with CNV in a subject, the method comprising administering to the eye of the subject a therapeutically effective amount of activated protein C (APC), thereby providing therapy to the subject.

In accordance with the present disclosure, APC comprises wild-type sequence of human APC, a functional partial sequence of APC, a derivative of APC or of a functional partial sequence thereof, or a variant of APC or of a functional partial sequence thereof.

In some embodiments, a disclosed method comprises administering to the eye of the subject a therapeutically effective amount of a variant of activated protein C (APC) or a functional partial sequence thereof.

In some embodiments, the therapy is treatment of retinal leakage and CNV associated with an ocular disease, disorder or condition. The ocular disease, disorder or condition may be characterized by being at least one of: caused directly by CVN, featuring development of CNV as a secondary stage or a complication thereof, or featuring CNV as a synchronous or asynchronous sequela thereof.

In some embodiments, the disease is neovascular age-related macular degeneration (nAMD), myopathy or angioid streaks.

In a further aspect, the present disclosure relates to an ophthalmic composition comprising a variant of activated protein C (APC) or a functional partial sequence thereof, and a carrier acceptable for ophthalmic application.

The APC variant may be one or more of: 3K3A-APC, RR229/230AA-APC, 5A-APC, APC-2Cys, K193E-APC, E149A-APC, a wild type APC in which residue 158 (Asp) is substituted with a non-acidic amino acid, or residue 154 (His) is substituted with an amino acid residue selected from the group consisting of Lys, Arg or Leu.

In some embodiments, the APC variant is 3K3A-APC.

In some embodiments, APC and/or APC variant is used in combined therapy with one or more active agents selected from anti-angiogenesis, anti-inflammatory, anti-bacterial, immunosuppressive, anti PDGF, anti-fungal and anti-viral agent.

### BRIEF DESCRIPTION OF THE DRAWINGS

The patent or application file contains at least one drawing executed in color. Copies of this patent or patent application publication with color drawings will be provided by the Office upon request and payment of the necessary fee.

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIGS. 1A-1C are Background art schemes of the blood retinal barriers (BRBs) (1A), a normal structure of the retina (1B), and retina featuring choroidal neovascularization in age related macular degeneration (AMD) (1C):

FIGS. 2A-2D are 3-dimensional confocal microscope images of retinal pigment epithelium cells (ARPE-19) stained with rabbit anti-ZO-1 antibody (bright) and NucBlue® (nuclear staining; light, round circular areas), following treatment with 0.0 μg/ml APC (control, 2A); 0.1 μg/ml APC (2B): 1 μg/ml APC (2C); or 10 μg/ml APC (2D);

FIG. 3 is a bar graph showing the FITC-dextran permeability (as % of control) of ARPE-19 cells treated with 0.0, 0.1 or 1 μg/ml APC (average results of 4 experiments);

FIG. 4 is a bar graph showing choroid blood vessel area of flattened choroid excised from mice 7 days after they underwent CNV induction by laser photocoagulation immediately followed by intravitreal injection of either 1 μl saline, 1 μg/μl APC or 25 μg/μl bevacizumab (5 mice in each group). Data are expressed as percent of control (laser application with saline treatment considered 100%). Comparisons vs. 100% were performed using one-sample two-tailed Student's t test. Comparisons between the bevacizumab and APC treatment were performed using unpaired two-tailed Student's t test. *P<0.05, **P<0.01 vs. Laser;

FIGS. 5A-5B are bar graphs showing volume (5A) and depth (5B) of newly formed choroidal blood vessels of flattened choroid-RPE sections following laser photocoagulation and intravitreal injections of saline, or APC (1 μg/animal). Neo vascularization was assessed by fluorescently by dextran perfusion test;

FIGS. 6A-6C are collective Z stack images (1 μm distance; 6A-6B), and a bar graph (6C) of exemplary flattened entire retinas following laser photocoagulation and intravitreal injections of saline (6A) or APC (1 μg/animal; 6B). Blood vessels are shown bright in the images. Choroidal blood vessels penetration area in the retinal sections is quantified in FIG. 6C;

FIGS. 7A-7B are bar graphs showing the effect of the APC mutant 3K3A-APC on the depth (7A), and volume (7B) of invading choroidal blood vessels in choroidal flat-mounts of mice induced to develop CNV by laser photocoagulation. Results obtained from 3 groups of mice (n=7-9 mice in each group) are presented: (i) mice treated with laser application immediately followed by intravitreal injection of saline; (ii) mice treated with laser application immediately followed by intravitreal injection of 1 μg/μl 3K3A-APC; and (iii) mice not treated with laser or 3K3A-APC (control). Seven days later, blood vessels were stained using FITC-dextran perfusion, choroidal flatmounts were isolated, and 3 dimensional confocal images were scanned. The data are presented in FIGS. 7A-7B as mean±SD;

FIG. 8 is a bar graph showing quantitative assessment of retinal leakage following CNV induction in mice by laser photocoagulation. Mice, which were each subjected to 3 laser applications, were then immediately injected intravitreally with the APC mutant 3K3A-APC (1 μg/μl) or saline (n=7 mice per group), and 7 days post laser application, leakage was measured by fluorescein angiography (FA);

FIG. 9 is a bar graph showing quantitative assessment of the protective effect of APC following Tie2 inhibition in mice. Mice were induced for CNV by laser applications in absence or presence of prior treatment with Tie2 kinase inhibitor, and then immediately injected intravitreally with APC or saline (n=5 mice per group). The control group did not receive any treatment. *P<0.05, ***P<0.001 vs. Control; ###P<0.01 vs. Laser+Salie;

FIGS. 10A-10D are bar graphs showing quantitative assessment of vascular endothelial growth factor (VEGF) and CNV volume and depth in flat RPE-choroid specimens form eyes of 3 groups of mice (7-9 mice per group): mice which developed CNV following laser photocoagulation (verified by fluorescein angiography (FA)) and treated with 1 μg/μl/mouse APC; mice which developed CVN and treated with saline; and mice without laser application, injected with saline (control). CNV was stained using FITC-dextran perfusion, and flat RPE-choroid specimens were immunostained with anti-VEGF antibodies. 10A-10B: VEGF volume (μm³) and depth (μm), respectively; 10C-10D: CNV volume (μm³) and depth (μm), respectively. The data are presented as mean±SD;

FIGS. 11A-11C are images of laser lesion sites (11A, 11B) and a bar graph (11C) showing the longitudinal effect of APC on VEGF levels in CNV lesion sites induced in mice subjected to laser photocoagulation (laser application) and immediately afterwards intravitreally injected with a single dose of APC (1 μg/μl/mouse) or saline. Mice not treated with laser application were injected with saline and served as control. Sequential cryosections were stained with anti-VEGF antibodies (red), and cell nuclei were stained with DAPI (blue). Representative histological section images of laser lesion sites from mice treated with saline (11A), or APC (11B) were taken 3 days post-treatment. Images of every fifth slide of each eye were captured digitally under the same settings, and VEGF staining was scored from 0-3 (light to heavy, respectively) Twenty images/group were used for analysis and the mean scoring of VEGF staining in laser lesion sites was calculated (11C). GCL: ganglion cells layer; INL: inner nuclear layer; ONL: outer nuclear layer. The data are presented as mean SD and analyzed using unpaired two-tailed Student's t test (the resulting P values were adjusted for four time points: 1, 3, 14 and 30 days, using Sidak's correction); and

FIGS. 12A-12H are immunofluorescent images of laser lesion sites (12A-12F) and bar graphs (12G-12H) showing the effect of 3K3A-APC on VEGF levels in CNV lesion sites induced in mice subjected to laser photocoagulation and immediately afterwards intravitreally injected with a single dose of 3K3A-APC (1 μg/μl/mouse) or saline. Mice not treated with laser application were injected with saline and served as control (7-9 mice in each group). Three days after CNV was verified, blood vessels were stained using FITC-dextran perfusion, flat RPE-choroid specimens were isolated and stained with anti-VEGF antibodies. Immunofluorescent images of RPE-choroid flatmounts present upper view (12A-12C), and Z-plane (12D-12F) of laser-induced CNV lesion (scale bar represents 50 μm and 30 μm for upper view and Z-plane, respectively). The arrow shows the Z plane (depth), asterisk point VEGF hexagonal staining of the RPE membrane. The volume (μm³) (12G) and depth (μm) (12H) of VEGF was quantified in eyes not treated with laser application (control), eyes subjected to laser and treated with saline (laser+saline) and eyes subjected to laser and treated with 3K3A-APC (laser+3K3A-APC). The data are presented as mean SD and analyzed using one-way ANOVA followed by Tukey post hoc test.

### DETAILED DESCRIPTION

Aspects of disclosed embodiments relate to the use of activated protein C (APC) and APC variants, i.e., mutated APC, in the therapy of neovascularization and retinal leakage, for example, neovascularization and retinal leakage related to, or associated with various ocular diseases.

Retinal pigment epithelium (RPE) is a brown monolayer of cells of the retina situated next to the choroid and composed of cells joined by tight junctions and filled with pigment, mainly melanin and lipofuscin. Depending upon the amount of pigment, the fundus will appear dark or light. The main functions of the RPE include control of the flow of fluid and nutrients entering the retina, absorption of scattered light, visual pigment metabolism, vitamin A metabolism which contributes to visual pigment regeneration, ingestion and digestion of photoreceptor discs (phagocytosis), retinal adhesion and synthesis of growth factors of adjacent tissues.

Optimal retinal function requires an appropriate, tightly regulated environment. Such regulation is ascertained by cellular barriers created by tight junction proteins, which separate and/or segregate functional compartments, maintain their homeostasis, and control transport between them. Exemplary tight junction proteins include Tie2, a transmembrane endothelial tyrosine kinase receptor that has been shown to protect endothelial and epithelial barrier functions (Minhas et al., 2010, FASEB J. 24:873-881), and occludin or claudins that bind to the 225-kDa cytoplasmic phosphoprotein zona occludens (ZO)-1, which links to the cytoskeleton and thereby provides junctional stability. Disruption of ZO-1 may lead to breakdown of tight junctions and an increase in vascular permeability. Blood retinal barriers (BRBs), particularly the outer barrier composed of RPE, and the inner barrier between retinal microvascular endothelium, are highly dynamic structures, capable of rapidly responding to physiological requirements as well as to changing extrinsic conditions.

Most of the retinal diseases involve vascular leakage and breakage of the inner or outer BRBs. There are two types of neovascularization that occur in the retina and cause vision loss: retinal neovascularization (RNV) in which new vessels sprout from the retinal capillaries and invade the vitreous and neural retinal layers, and choroidal neovascularization (CNV), in which new vessels sprout from the choroidal vasculature and invade the sub-retinal space.

Destructing or inhibiting choroidal invading blood vessels with anti-vascular endothelial growth factor (VEGF) agents (e.g., Bevacizumab, Ranibizumab, Aflibercept), and laser coagulation of the blood vessels are the main state-of art treatments for CNV. However, these treatment methodologies often damage the retina, destroy needed blood vessels and/or cause damaging retinal scars, yet neither of these treatments solve the underlying problems that cause the blood vessels to invade. In addition, reduced therapeutic response to anti-VEGF agents most often follows repeated administration over time. Thus, relapses are common and many patients still lose vision within a year or two.

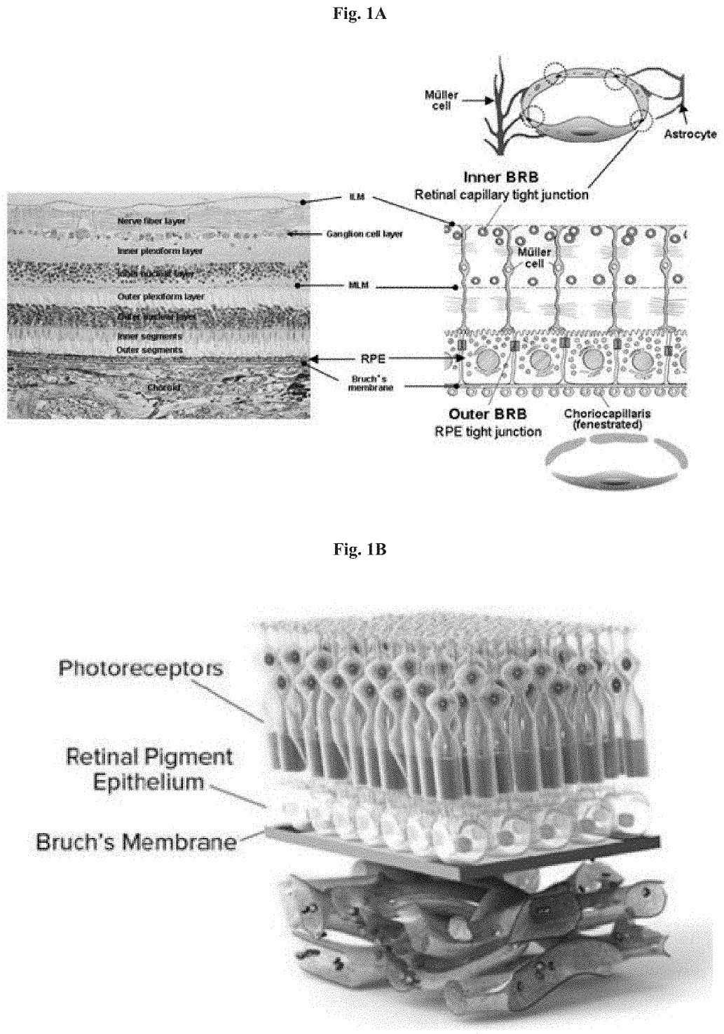

For example, neovascular age-related macular degeneration (nAMD, also known as wet AMD) which commonly causes vision loss from aberrant angiogenesis, is characterized by the growth of new, immature choroidal blood vessels, through Bruch's membrane following breaking through the outer-BRB and growing into the sensory retina under the macula, where they can leak fluid or hemorrhage under the retina (see, Background Art FIGS. 1A-1C). This disease is one of the leading causes of visual impairment in industrialized countries. Despite the clinical benefits achieved with VEGF suppression, there are many patients who have a suboptimal response. CNV is the leading cause of severe vision loss in various other ocular diseases such as, but not limited to, angioid streaks and high myopia. Treatment directed against the cause of the disease yet remains difficult to accomplish, as the underlying etiology is very complex and elusive. However, developing new agents, in addition to anti-VEGF agents, may afford alternative pathways in prevention and treatment of CNV.

Activated protein C (APC) is a plasma serine protease with, inter alia, endothelial and epithelial barrier protective properties.

In search for treatment modalities that would meet the yet continuing need for treatment and prevention of CNV and/or retinal leakage, the present inventors have envisaged a barrier protective effect of APC on impaired adhesions in critical junctions of BrM and/or RPE in the retina, which would result in prevention or at least amelioration of retinal leakage and CNV.

The present inventors have devised in vitro and in vivo models to assess the protective effect of APC on RPE and BRBs, and successfully practiced a novel method of treating CNV based on intravitreal administration of APC.

As demonstrated in the Examples section that follows, in an in vitro model of human retinal pigment epithelial cells (ARPE-19), APC induced translocation of tight junction protein Zonula Occludens 1 (ZO-1) to the ARPE19 cell membrane and reduced RPE permeability of labeled dextran as compared to untreated cells (see, Examples 1 and 2 herein). In an in vivo model devised by the present inventors, CNV was induced by indirect diode laser photocoagulation on male C57BL/6J mice. Intravitreal injection of APC (1 μg/animal) immediately following injury, dramatically reduced CNV area (Example 3, herein) as well as CNV volume and depth, as shown by 3-dimensional analysis (see, Example 4 herein). The APC effect was comparable to the effect of bevacizumab, the current treatment of choice for CNV.

The present inventors have further quantitated blood vessels formation following laser injury in mice and have successfully demonstrated that APC reduced significantly the development and penetration of blood vassals from the choroid into the retina (see, Example 5 herein).

As APC is a natural coagulation inhibitor, bleeding risk needs to be taken into consideration. For example, it has been shown that APC therapy increased risk for serious bleeding events in adult patients with severe sepsis. The APC mutant 3K3A-APC is a recombinant engineered variant of APC (3 Lysine residues replace 3 Alanine residues) with markedly reduced anticoagulant activity. Although the replacement of the 3 residues in the 3K3A-APC variant reduce APC's interactions with its substrate clotting factor Va, it does not affect APC's interactions with its cell receptors, including binding to endothelial protein C receptor and activating protease-activated receptors (PAR) 1 and 3 (Griffin at al., 2015, Blood, 125:2898-907; Griffin at al., 2018, Blood, 132:159-169; Mosnier et al., 2004, Blood, 104:1740-4). Thus, both wild-type APC and 3K3A-APC have anti-apoptotic, anti-inflammatory, neuroprotective, cytoprotective, and endothelial barrier stabilization properties.

Using the in vivo model of CNV designed by the present inventors, they have evaluated the ability of 3K3A-APC to induce regression of CNV. Thus, CNV was induced by laser photocoagulation on C57BL/6J mice, and 3K3A-APC was injected intravitreally after verification of CNV presence. CNV volume and vascular penetration were evaluated on RPE-choroid flatmount by FITC-dextran imaging. As shown in Example 6 herein, 3K3A-APC induced regression of pre-existing CNV. In addition, the resent inventors have successfully shown that VEGF levels, measured in the CNV lesion sites, significantly decreased upon APC and 3K3-APC treatment. Reduction in VEGF was sustained 14 days post a single APC injection (see Examples 9-11 herein).

To evaluate the potential clinical use of APC, the present inventors simulated the clinical setting, whereas pathological leakage is already present in most of the patients diagnosed with CNV. They have applied treatment just after confirming the presence of leakage from CNV by fluorescein angiography (FA), and demonstrated that, similarly to APC, 3K3A-APC induced CNV regression (see Example 7 herein). These results suggest that the anticoagulant properties of APC are not mandatory for its beneficial effects on the retina. Blood vessels involved in the pathogenesis of CNV are immature, lack structural integrity, and leak fluid, leading to hemorrhage and exudates, accompanied by fibrosis and loss of vision. Therefore, the present inventors have shown, for the first time, that APC variants such as 3K3A-APC may prevent exposure of the intraocular environment to the anticoagulant properties of APC, thereby minimize or event completely prevent a risk for retinal hemorrhage.

In one aspect, the present disclosure relates to a method for preventing or treating retinal leakage and/or choroidal neovascularization in a subject, the method comprising administering to the eye of the subject a therapeutically effective amount of activated protein C.

A disclosed method is applicable in treatment and/prevention of a disease, disorder or condition in which CNV and/or retinal leakage associated with CNV are objective indications, e.g., signs or manifestations. Embodiments disclosed herein relate to therapy (i.e., curing, ameliorating, reducing and even preventing) of CVN and/or retinal leakage, e.g., due to CNV, associated with an ocular disease, disorder or condition.

“Activated protein U” as used herein is to be interpreted in a broad manner so as to encompass various forms of APC known in the art and those yet to be discovered. Non-limiting examples include: wild-type sequence of human APC; a polymorphic variant of human APC; an interspecies homolog of human APC; a functional partial sequence of wild-type human APC or of its polymorphic variant or interspecies homolog; a derivative of wild-type human APC or of its polymorphic variant, interspecies homolog or functional partial sequence; and a variant (mutant) of wild type human APC or of its polymorphic variant, interspecies homolog, or functional partial sequence. The term “administration of APC” as used herein refers to administration of one or more of these forms of APC, either combined in a single dosage form or administered consecutively in one or more separate dosage forms, each comprising one or more forms of APC as described herein.

In some embodiments, the APC is wild type APC as defined herein and/or or a functional partial sequence thereof.

In some embodiments, the APC is an APC variant as defined herein and/or a functional partial sequence thereof.

In some embodiments, the APC is a mixture of wild type APC and at least one variant thereof and/or functional partial sequences thereof.

The term “CNV associated with an ocular disease, disorder or condition”, as used herein, refers to CVN caused directly by a disease, disorder or condition, or CNV development is a feature of a disease, disorder or condition, for example, a disease, disorder or condition in which CNV is a secondary stage or a complication thereof and, optionally, become worsen, exacerbates or progressed because of the CNV process. CNV being featured as a synchronous or asynchronous sequela of a disease, disorder or condition, namely CNV resulting from earlier occurrence of such diseases or disorders, for example infectious or noninfectious diseases, is also regarded herein as CNV associated with an ocular disease, disorder or condition.

Thus, in an aspect of some embodiments, a disclosed method is useful for therapy of ocular disease, disorder or condition characterized by being at least one of: caused directly by CVN, featuring development of CNV as a secondary stage or a complication thereof, or featuring CNV as a synchronous or asynchronous sequela thereof.

Ocular diseases associated with CNV, treatable by the method described herein include, but are not limited to, age-related macular degeneration (AMD), particularly, nAMD (wet AMD), pathologic myopia, and pseudoxanthoma elasticum with angioid streaks.

CNV and blood leakage form sprouting, immature choroidal blood vessels in the retina can result from a myriad of inflammatory and non-inflammatory diseases of the choroid and the retina, such as, but not limited to, optic neurtis, and other inflammatory diseases of the optic nerve such as papilledema, anterior and ischemic optic neuropath (AION), blood vessels occlusion, Behçet's disease and diabetes complications such as retinopathy.

CNV can be the sequela of both infectious and noninfectious uveitis. In the infectious diseases, Toxoplasmosis (caused by the parasite Toxoplasma gondii and accounts for toxoplasmic retinochoroiditis), Toxocara canis, Tuberculosis, and viral retinopathies, can have CNV as synchronous or asynchronous sequela. Noninfectious uveitis associated with CNV includes, but is not limited to, punctate inner choroidopathy (PIC), multifocal choroiditis (MFC), acute posterior multifocal placoid pigment epitheliopathy (APMPPE), serpiginous choroiditis (SC), presumed ocular histoplasmosis syndrome (POHS) and Vogt-Koyanagi-Harada (VKH) disease.

For example, CNV is commonly associated with MFC, which is found in 32% to 46% of patients, and in POSH, the primary cause of visual impairment is the occurrence of CNV in the macula, which results in exudation and subsequent scarring.

Bacteria can also affect the eye, and CNV can (although rarely) be of the most severe sequela. The retinal colonization can occur by metastasizing the choroid during endocarditis, aortic valve infection, renal and bone abscess and intravenous drug abuse. The choroidal neovascular membrane typically grows near an active or quiescent choroidal granuloma. Classic CNV is generally the typical occurrence in bacterial endocarditis, which grows close to the primary chorioretinal lesion, or in neighboring area of an old atrophic scar.

Viruses have also been associated with CNV, predominantly as a late complication (Neri et al., 2009, Middle East Afr J Ophthalmol., 16(4): 245-251), for example, the West Nile virus can cause an extensive ischemic capillaropathy in the macula that may develop into a choroidal neovascularization near a chorioretinal scar.

CNV can be a late sequela of Fungi, such as Candida albicans, Cryptococcus neoformans and Aspergillus fumigatus, that have been described as potential pathogens for the eye.

Ocular disorders associated with CNV, which can benefit from treatment with APC or a variant thereof in accordance with embodiments of the method described herein include, but are not limited to, oxidative damage, drusen biogenesis, lipofuscin accumulation, abnormalities of Bruch's membrane, vascular changes in the eye that impede regulation of blood pressure and flow, thus, limiting the exchange of nutrients and removal of metabolic waste and creating conditions of ischemia, physiologic aging, genetic factors (mutations in the complement pathway) and environmental factors (for example, smoking, irradiation, lack of vitamins and the like). For example, CNV may be secondary, originating from an old chorioretinal scar.

“Conditions associated with CNV” as defined herein refer to accidental, occasional, one-time incidences in which CNV develops as a result of, for example, traumatic injury of the retina, complications during an ophthalmic medical procedure such as surgery, laser treatment or routine checkup, and the like.

The terms “therapy”, “treatment”, “treating”, “treat” as used herein are interchangeable and refer to: (a) preventing a disease, disorder, or condition from occurring in a human which may be predisposed to the disease, disorder and/or condition but has not yet been diagnosed as having it; (b) inhibiting the disease, disorder, or condition, i.e., arresting its development; (c) relieving, alleviating or ameliorating the disease, disorder, or condition, i.e., causing regression of the disease disorder and/or condition; and (d) curing the disease, disorder, or condition. In other words, the terms “therapy”, “treat,” “treatment,” and “treating,” extend to prophylaxis, namely, “prevent,” “prevention,” and “preventing,” as well as treatment per se of established conditions. Accordingly, use of the terms “prevent,” “prevention,” and “preventing,” would be an administration of the active agent to a person who has in the past suffered from the aforementioned conditions, such as, for example, retinal leakage or CNV, but is not suffering from the conditions at the moment of the composition's administration.

Thus, the terms “treatment”, “therapy” and the like include, but are not limited to, changes in the recipient's status. The changes can be either subjective or objective and can relate to features such as symptoms or signs of the disease, disorder or condition being treated. For example, if the patient notes improvement in visual acuity, reduced central visual field defects or decreased pain, then successful treatment has occurred. Similarly, if the clinician notes objective changes, such as by fluorescein angiography (FA), indocyanine green angiography (ICGA) or optical coherence tomography (OCT), then treatment has also been successful. Alternatively, the clinician may note a decrease in the size of lesions or other abnormalities upon examination of the patient (for example, grayish-white subretinal changes together with retinal edema, hard exudations, and reduced subretinal and intraretinal hemorrhage). This would also represent an improvement or a successful treatment. Preventing the deterioration of a recipient's status is also included by the term. Therapeutic benefit includes any of a number of subjective or objective factors indicating a desirable response of the condition being treated as discussed herein

In some embodiments, the method of treating or preventing ocular diseases, disorder or conditions may include administrating to a subject in need thereof an effective amount of APC, a variant of APC or a functional partial sequence thereof of from about 0.1 μg/μl to about 50 μg/μl, for example, from about 0.1 μg/μl to about 0.5 μg/μl, from about 0.1 μg/μl to about 1 μg/μl, from about 0.5 μg/μl to about 1 μg/μl, from about 0.5 μg/pI to about 2 μg/μl, from about 1 μg/μl to about 5 μg/μl, from about 2 μg/μl to about 8 μg/μl, from about 5 μg/μl to about 10 μg/μl, from about 11 μg/μl to about 15 μg/μl, from about 12 μg/μl to about 20 μg/μl, from about 15 μg/μl to about 20 μg/μl, from about 15 μg/μl to about 25 μg/μl, from about 20 μg/μl to about 28 μg/μl, from about 25 μg/μl to about 35 μg/μl, from about 30 μg/μl to about 40 μg/μl, from about 35 μg/μl to about 45 μg/μl, or from about 40 μg/μl to about 50 μg/μl, including any subranges and individual values therebetween.

In some embodiments described herein, application of APC, APC variant or a functional partial sequence thereof in the treatment of CNV is affected by intravitreal administration, for example intravitreal injection of the active agent(s).

Optionally, in accordance with these embodiments, while taking into consideration that fluids in the retina (vitreous) dilute APC administered in any of the effective amounts described herein by a factor of 20-50, a contemplated ophthalmic composition, in accordance with an aspect of the present disclosure, formulated, e.g., as intraocular irrigating solution, will comprise a concentration of APC, APC variant or a functional partial sequence thereof which is 20-50 times higher than any of the effective concentration ranges described herein. Optionally, eye drops, for example of 0.3% levofloxacine, are applied immediately after injection.

In some embodiments, APC, APC variant or a functional partial sequence thereof is topically applied to the eye.

The method of treating CNV and vascular leakage in the retina as described herein may benefit, for example generate a synergistic effect, when combined with other treatment modalities, particularly treatment modalities that address the prime motive or the trigger for development of CNV.

Taking into consideration that chronic subclinical inflammation and clinical inflammation of the retina and the choroid can be the basis for the pathogenesis of CNV and thereby account for leakage in the retina, in some embodiments featuring a combined therapy, the treatment strategy for CNV secondary to noninfectious inflammations would be directed at controlling the inflammatory process. Accordingly, systemic medications, for example, corticosteroids such as dexamethasone (e.g., Ozurdex) and derivatives thereof, with or without immunosuppressive agents may be indicated along with APC and/or APC variant. Additionally, or alternatively, therapies aimed directly at the neovascular process, such as any of the intravitreal anti-VEGF agents, are indicated, particularly when the anti-inflammatory therapy shows an insufficient response. For example, in embodiments where CNV is associated with recurrent retinochoroiditis, the classical association of anti-toxoplasmic antibiotics with corticosteroids may be given to a patient, optionally, together with anti-VEGF therapy. Combined therapy comprising anti-inflammatory and anti-VEGF therapy aimed directly at the neovascular process is recommended in severe cases. Non-limiting examples of anti-VEGF agents that can be used in a combined treatment modality include bevacizumab, ranibizumab and aflibercept.

VEGF is expressed within the normal retina even in absence of active angiogenesis, acting as an important neurotrophic factor crucial for survival and normal function of the retina. In the aging eye, the pathologic level of VEGF released by the RPE is a detrimental factor in developing CNV and neovascular age-related macular degeneration (nAMD; also called wet AMD). The current efficacious treatments for CNV are mostly based on anti-VEGF agents, yet, suboptimal response, slow loss of efficacy, and long-term concerns regarding the continuity of the neurodegenerative process restricted their full success. Using the laser-induced CNV mice model, the present inventors demonstrated that the enhanced expression of VEGF, detected in the mice eyes with CNV, was significantly reduced upon APC or 3K3A-APC treatment. Moreover, VEGF is known to be secreted in a polarized manner from the basolateral aspect of the RPE toward the choriocapillaris layer. The present inventors scanned the VEGF gradient from the RPE toward the choriocapillaris and found that VEGF expression was detected all over RPE-choroid specimen in eyes with CNV that were treated with saline; however, treatment with APC and 3K3A-APC reversed the expanded expression of VEGF and restricted it to the RPE edge.

Interestingly, the APC-induced reduction in VEGF expression in CNV lesions sites was sustained up to 2 weeks post single injection (see Examples 9-11 herein). These findings for 3K3A-APC support other findings by the present inventors showing a reduction in CNV two weeks post a single dose of APC treatment (Livnat et al., Exp Eye Res. 2019, 186:107695), and clearly show that VEGF reduction mediates the protective effects of APC and 3K3A-APC in CNV.

APC and APC variants interact with cellular receptors such as protease-activated receptor (PAR)1, PAR3, endothelial protein C receptor (EPCR) and others, and induce signal transduction that leads to barrier protection and stabilization. Anti-VEGF agents, on the other hand, bind to VEGF and inhibit its ability to bind and activate VEGF receptors. In view of these different modes of action, combined treatment featuring co-administration of one or more anti-VEGEF agents and APC and/or APC variant may have synergistic effects in treating or preventing CNV. In some embodiments, use of APC/APC variant as the sole active agent in treatment of CNV is applied to patients resistant to anti-VEGF treatment.

In some exemplary embodiments, when CNV is secondary to an autoinflammatory disorder, APC and/or APC variant can be administered to a patient tougher with immunosuppressive agents, for example, an association of steroids, cyclosporine A and, in some cases, azathioprine. The steroids may be periocular or systemic steroids.

In some exemplary embodiments, in idiopathic choroidal neovascularization (ICNV), where CNV is the only reliable finding in the retina and no other abnormalities are detectable, APC/APC variant treatment can be combined with anti-inflammatory medications.

Since the inflammatory process is not only loco-regional and the whole immune system appears to be involved, the use of systemic steroids should be always considered. The safety and efficacy of immunosuppression for the control of choroidal new vessels are known. The choice of the immunosuppressant should be established on the basis of the characteristics of the drug itself. For example, mycophenolate mofetil (MMF) can be the choice of drug for the long-term control of inflammatory CNV since it has proven to be effective in improving arteriolopathy and decreasing the amount of soluble mediators involved in CNV pathophysiology.

Platelet derived growth factor (PDGF) plays an important role in the angiogenesis cascade that is activated in retinochoroidal vascular diseases. One possible explanation for the involvement of PDGF in the CNV process includes entry of platelets and monocytes into the vitreous and subretinal space upon injury to the blood-retina barrier, with subsequent platelet aggregation and PDGF discharge. Interleukins such as IL-1 and TGF-B, released from activated macrophages may lead to further synthesis of PDGF.

In some embodiments of combined therapy in accordance with the present disclosure, APC/APC variant may be co-administered with anti-PDGF agents that block the effects of PDGF in the angiogenesis process in the retina. Non-limiting examples of anti-PDGF agents include PDGF antagonists, such as designed ankyrin repeat protein (DARPin) that selectively binds to, and antagonizes PDGF-BB in subretinal CNV, optionally co-administered with a protein that antagonized VEGF-A (anti-VEGF protein). A further exemplary high affinity PDGF antagonist is E10030, which binds to PDGF and blocks its binding to PDGFR-β. This antagonist has increased effectiveness when co-administered with an anti-VEGF agent such as ranibizumab.

In some embodiments of the combined therapy, APC/APC variant is provided to a patient together with one or more treatments selected from anti-angiogenesis, anti-inflammatory, anti-bacterial, immunosuppressive, anti PDGF, anti-fungal and viral therapies. In some embodiments, the co-administered active agent or drug is administered together with APC and/or APC variant in a single dosage form, optionally by intravitreal injection. Additionally, or alternatively, the co-administered active agent or drug is administered in one or more separate dosage forms, either before, simultaneously with, or subsequently after administration of APC. In some embodiments, the co-administered active agent is administered systemically. Alternatively, or additionally the co-administered active agent is administered locally, optionally by intravitreal injection or topical application.

The regimen of APC/APC variant administration in embodiments described herein, is dictated by various considerations such as the state of the retina, the progress of healing, tolerance of the patient and the like. For example, a single dose may be applied once a month or once a week for up to 6 to 8 weeks, wherein the gap between successive administrations and necessity of continuing APC administration is determined based on the state of the treated retina, progression of healing and side effects evaluated.

### Pharmaceutical Compositions

In an aspect of some embodiments, there is provided a pharmaceutical composition comprising, as the active agent, APC, a functional fragment of APC, an APC derivative, an APC variant (mutant), an APC homolog or a combination thereof, for use in treatment of CNV and/or retinal leakage resulting thereform.

In some embodiments, the pharmaceutical composition described herein is used in treatment of ocular diseases and disorder that are associated with, or caused by CNV, such as, but not limited to, nAMD (wet AMD), angioid streaks and high myopia.

The pharmaceutical compositions described herein may comprise, besides the active agent APC, a pharmaceutically acceptable carrier and excipients and optionally further comprise chemical components which, for example, facilitate sustained release of the active agent in the treated eye.

Accordingly, in any of the methods and uses described herein, wild type APC or any of its functional partial sequences (fragments), derivatives, variants and homologs described herein can be provided to an individual either as is, or as part of a pharmaceutical composition where it is mixed with a pharmaceutically acceptable carrier.

In some embodiments, an ophthalmic composition is provided, comprising a variant of APC or a functional partial sequence thereof, and a carrier acceptable for ophthalmic application.

As used herein a “pharmaceutical composition” refers to a preparation of one or more of APC, a derivative, variant or a homologs thereof and/or a functional fragment thereof as described herein (as active ingredient), or physiologically acceptable salts or prodrugs thereof, with other chemical components including but not limited to physiologically suitable carriers, excipients, lubricants, buffering agents, antibacterial agents, bulking agents (e.g., mannitol), antioxidants (e.g., ascorbic acid or sodium bisulfite), anti-inflammatory agents, anti-viral agents, chemotherapeutic agents, anti-histamines and the like. The purpose of a pharmaceutical composition is to facilitate administration of a compound to a subject. The term “active ingredient” refers to a compound, which is accountable for a biological effect. An ophthalmic composition is one embodiment of a pharmaceutical composition.

As used herein, the terms “physiologically acceptable” and “acceptable carrier” mean approved by a regulatory agency of the Federal or a state government or listed in the U.S. Pharmacopeia or other generally recognized pharmacopeia for use in animals, and more particularly in humans. Herein, the phrase “physiologically suitable carrier” refers to an approved carrier or a diluent that does not cause significant irritation to an organism and does not abrogate the biological activity and properties of a possible active agent.

Herein the term “excipient” refers to an inert substance added to a pharmaceutical composition to further facilitate processes and administration of the active ingredients. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention, thus, may be formulated in conventional manner using one or more pharmaceutically acceptable carriers comprising excipients and auxiliaries, which facilitate processing of the compounds into preparations which can be used ophthalmologically. Proper formulation is dependent upon the route of administration chosen. The dosage may vary depending upon the dosage form employed and the route of administration utilized. The exact formulation, route of administration and dosage can be chosen by the individual physician in view of the patient's condition.

The pharmaceutical composition may be formulated for administration in either one or more of local routes depending on the area to be treated.

In some embodiment, the pharmaceutical composition is formulated in a form suitable for intravitreal administration. In some embodiments, the pharmaceutical composition is formulated in a form suitable for topical application on the applied area.

According to an embodiment, the pharmaceutical composition described hereinabove is packaged in a packaging material and identified in print, in or on the packaging material, for use in the treatment of a medical condition associated with CNV.

### Activate Protein C (APC)

Protein C may be purified from clotting factor concentrates or from plasma by well-known methods, and then activated in vitro to obtain the activated form APC, however such processes are complex and expensive, in part due to the limited availability of the starting material and the low concentration of protein C in plasma. Furthermore, the therapeutic use of products derived from human blood carries the risk of disease transmission. Thus, in some embodiments, the APC used in contemplated therapeutic applications is a commercially available human recombinant wile-type APC, a functional partial sequence thereof, a derivative thereof and/or a variant (mutant) thereof, obtained by known genetic engineering techniques, such as recombinant DNA techniques.

In some embodiments, the full-length, wild-type human recombinant APC is applied for treatment of CNV. Alternatively, or additionally, a polymorphic variant, a mutated APC, or an interspecies homolog of human APC is applied.

In some embodiments, a functional fragment of wild-type human APC is applied in therapy of ocular diseases, disorders or conditions associated with CNV. As defined herein “a functional fragment”, is a partial sequence of the wild type APC protein, or of a variant of APC, or of an interspecies homolog thereof, that retains the biological activity of the whole or intact protein. The functional fragment can comprise up to 95%, up to 90%, up to 85%, up to 80%, up to 75% or even less, of the APC amino acid sequence, and it maintains wild type or near wild type APC functionality, or variant or near variant functionality, or homolog or near homolog functionality.

In some embodiments, a derivative of the wild-type APC or of a functional partial sequence thereof is applied for therapeutic purposes. As used herein, “a derivative of APC” encompasses wild-type APC, a polymorphic variant and an interspecies homolog thereof, or any functional partial sequence thereof, in which the amino acid sequence has been modified post protein synthesis, having substantially the same biological activity as wild-type APC. The phrase “having substantially the same biological activity” as used herein refers to APC derivatives having about 60% identical, about 65% identical, about 70% identical, about 75% identical, about 80% identical, about 85% identical, about 90% identical, about 95% identical, or 100% identical biological activity as wild type human activated protein C.

Post synthesis modification of APC or functional partial sequence thereof comprises chemical or physical modifications, or both, of one or more amino acids. APC or a functional partial sequence thereof that have undergone a chemical or physical modification are also termed herein “a chemical derivative” and “a physical derivative”, respectively. For example, a derivative of APC may have amino acid sequence which is identical to the wild type sequence, but contains a post-synthesis conformation modification (i.e., a physical derivatization).

Examples of APC derivatives useful for the purpose of embodiment described herein are further discussed and disclosed, for example, in U.S. Pat. No. 5,516,650.

In some embodiments, a variant of the wild-type APC or of a functional partial sequence thereof is applied for therapeutic purposes. As used herein, the terms “a variant of APC” and “an APC mutant” are interchangeable and encompass wild-type APC, a polymorphic variant and an interspecies homolog thereof, or any functional partial sequence thereof, in which one or more of the naturally coded amino acids has been substituted and/or deleted via post translation modification. “A variant of APC” or “an APC mutant” further includes the naturally coded amino acids sequence containing additional one or more amino acids. Post translation substitution modification comprises replacement of one or more naturally coded amino acids of APC with one or more amino acids selected from natural and non-natural amino acids. Post translation addition modification comprises addition of one or more amino acids selected from natural and non-natural amino acids to the naturally coded amino acid sequence. Modification resulting in substitution, addition or deletion of one or more amino acid is also referred to herein as “biological derivatization”.

The term “natural amino acid” as used herein and in the art refers to the 20 naturally encoded and common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) as well as to pyrolysine and selenocysteine. “Non-natural amino acids”, as used herein, include, but are not limited to, amino acid analogs that function in a manner substantially similar to the naturally occurring amino acids. Amino acid analogs refer to compounds that have the same basic chemical structure as a naturally occurring amino acid, by way of example only, an α-carbon that is bound to a hydrogen, a carboxyl group, an amino group, and an R group. Such analogs may have modified R groups (by way of example, norleucine) or may have modified peptide backbones, while still retaining the same basic chemical structure as a naturally occurring amino acid. Non-limiting examples of amino acid analogs include homoserine, norleucine, methionine sulfoxide, methionine methyl sulfonium.

The term “non-natural amino acid” further includes naturally encoded amino acids (including but not limited to, the 20 common amino acids, pyrrolysine and selenocysteine) as well as amino acid analogs that have undergone chemical modifications. Non-limiting examples of chemically modified amino acids include N-acetylglucosaminyl-L-serine, N-acetylglucosaminyl-L-threonine, and O-phosphotyrosine.

Post-translation modification of the APC protein may include an “amino terminus modification group”, namely attachment of a molecule to the protein's terminal amine group. By way of example only, terminus modification groups include polyethylene glycol or serum albumin. Terminus modification groups may be used to modify therapeutic characteristics of APC, including but not limited to increasing the serum half-life of APC.

Substitution of a single natural amino acid or a small percentage of natural amino acids in the encoded APC sequence is considered herein a “conservatively modified variant” of APC where the alteration results in the substitution of a natural amino or a small percentage of natural amino acid with chemically similar (analog) amino acid(s).

In some embodiments, an APC variant comprises a substitution, addition or deletion or any combination thereof, of amino acids, which provide one or more of the following features to the protein: increased affinity for a receptor, increased stability, modified (e.g., increased) aqueous solubility, increased solubility in a host cell, modulated protease resistance, modulated serum half-life, reduced anticoagulant activity, modulated immunogenicity, and/or modulated expression relative to the wild-type APC. Modulating biological activity as used herein refers to increasing or decreasing the reactivity, altering the selectivity, and enhancing or decreasing the substrate selectivity of APC and any functional parts thereof.

Usually, the modifications affected have beneficial effects on APC, such as improving its stability and/or its biological activity, and/or reducing or eliminating un-desired activity, for example, reducing anticoagulant activity of APC thereby reducing the risk of bleeding. For example, variants of recombinant APC that have markedly reduced anticoagulant activity, but retain near normal anti-apoptotic (cytoprotective) activity, so that the ratio of anti-apoptotic to anticoagulant activity is greater in the variants than it is in wild-type or endogenous activated protein C. Non-limiting examples of such recombinant APC mutants are KKK191-193AAA-APC (substitution of lysine residues 191, 192 and 193 with alanine residues in a surface-exposed loop containing Lys191-193; also known in that are as “3K3A-APC”), RR229/230AA-APC (substitution of arginine residues 229 and 230 with alanine residues), and RR229/230AA plus KKK191-193AAA-APC, a combination of 3K3A and RR229/230AA-APC also known in the art as “5A-APC” (see, U.S. Pat. Nos. 9,192,657 and 7,489,305). Given their reduced anticoagulant activity, these exemplary APC variants provide significantly reduced risk of bleeding (variants 5A-APC and 3K3A-APC have <10% residual anticoagulant activity). 3K3A-APC has been reported to provide neuroprotection and extended therapeutic window (Griffin et al., 2015, Blood, 125:2898-2907). Other APC mutants that may be used in accordance with some embodiments include APC-2Cys, K193E-APC, and E149A-APC disclosed in Griffin et al. (supra), and APC variants that include the substitution of residue 158 (Asp) with a non-acidic amino acid residue such as Ala, Ser, Thr or Gly, or a substitution of residue 154 (His) with an amino acid residue such as Lys, Arg or Leu.

In some embodiments, the APC variant is 3K3A-APC or a functional partial sequence thereof.

A modified wild-type APC can feature a chemical derivatization, physical derivatization, a biological derivatization or any combination thereof. In some embodiments, such derivatizations are regioselective. In some embodiments, such derivatizations are regiospecific.

The amino acid sequence of a chemical, physical or biological derivative of APC can be at least 70%, at least 80%, at least 85%, at least 90%, at least 95%, at least 98%, or even 100% identical to the wild type APC sequence.

In some embodiments, the biological activity of any of the APC derivatives is improved by about 5%, about 10%, about 15%, about 20%, about 30%, about 50%, about 60%, about 70%, about 80%, about 85%, about 90%, about 95%, about 100%, about 110%, or more, including any intermediate values therebetween, compared to the biological activity of any of the wild type ACP or functional partial sequence thereof.

The form “modified or unmodified” means that the natural amino acid sequence being discussed is optionally modified, that is, the natural amino acid sequence of APC under discussion can be modified or unmodified

It is expected that during the life of a patent maturing from this application many relevant chemical, physical and biological derivatives of APC or of functional partial sequences thereof will be developed and the scope of the term “activated protein C, functional partial sequence thereof, derivative thereof or variant thereof” is intended to include all such new technologies a priori.

The terms “comprises”, “comprising”, “includes”, “including”, “having” and their conjugates mean “including but not limited to”.

The term “consisting of” means “including and limited to”

As used herein, the singular form “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Various embodiments and aspects as delineated hereinabove and as claimed in the claims section below find experimental support in the following examples.

### EXAMPLES

Reference is now made to the following examples, which together with the above descriptions illustrate some embodiments in a non-limiting fashion.

### Materials and Methods

### Animals

All animal experiments were performed according to the ARVO statement's guidelines for the Use of Animals in Ophthalmic and Visual Research and the approval of the Institutional Animal Care and Use Committee at Rabin Medical Center, Israel. Eight-week-old male C57BL/6J mice weighing 19 to 25 grams were purchased (Harlan Laboratories Ltd., Jerusalem, Israel or Envigo RMS, Israel) and handled according to recommendations of the hospital's Institutional Animal Care and Use Committee (i.e., the mice were kept in a constant temperature (20° C.-22° C.) with 12 hours light-dark cycles). For all animal experiments, animal allocation to treatments was randomized, and each experiment was repeated 2-3 times.

### Activated Protein C

Human APC was purchased from Haematologic Technologies Inc. USA (catalog #HCAPC-0080). Murine recombinant 3K3A-APC (KKK192-194AAA; 3 consecutive lysine residues 191-193 are replaced with 3 alanine residues) was prepared as described in Mosnier et al., 2004 (Mosnier et al., Blood 2004, 104:1740-4).

### Cell Culture.

Human retinal pigment epithelial (RPE) cells (ARPE-19 cell line) were purchased from ATCC (Manassas, Va.) and cultured under standard conditions according to the manufacturer's instructions. Growth media: DMEM/F-12 (HAM) 1:1 (Biological Industries, Israel, catalog #01-170-1A) was supplemented with 10% Fetal bovine serum (Biological Industries, Israel, catalog #04-121-1A), glutamine 1 mM (Biological Industries, Israel, catalog #03-022-1B), 100 u/ml penicillin, 0.1 mg/ml streptomycin and 12.5 μ/ml nystatin (Biological Industries, Israel, catalog #03-032-1B). Experiments were performed using passages 10-30 of the culture. For immunofluorescence assay, cells were plated at a concentration of 1.3×10⁴ cells/cm² and grown for a minimum of 30 days, and the media was exchanged every 2-3 days. Cells were washed with starvation medium (cells complete growth medium lacking serum), and treated with 0.1, 1.0, or 10 μg/ml activated protein C (APC) in the starvation medium for 10 minutes, after which the medium was replaced with complete growth medium and the cells were incubated for up to 4 hrs.

### In Vitro Permeability Assay.

Retinal pigment epithelium (RPE) cells (cells that form the pigmented layer of retina that nourishes retinal visual cells, and firmly attached to the underlying choroid) were plated at a concentration of 6.7×10³ cells/cm² on a 1 μm polyethylene terephthalate (PET) Transwell® insert (millicell, Millipore Corporation, Switzerland). The cells were grown for 30 days to confluency in 600 μl medium in the lower chamber and 100 μl in the upper chamber. Medium was exchanged every 2-3 days. Cells were then washed with starvation medium (basal medium containing 1 mM glutamine, 100 u/ml penicillin, 0.1 mg/ml streptomycin and 12.5 un/ml nystatin), and incubated with starvation medium containing 10.0, 1.0, 0.1 or 0.0 μg/ml APC. Following 10 minutes incubation with APC, the upper chamber medium was replaced with fluorescein isothiocyanate (FITC) dextran 70 kD, 1000 μg/ml (Sigma, Israel, catalog #FD-70) for up to 6 hours. The lower chamber medium was replaced with basal medium. Medium samples from the lower part of the insert were taken. FITC fluorescence, representing flow across the cell layer, was detected using a multi-detection microplate reader (Synergy™ HT, BioTek) at 485 nm excitation. Medium samples were returned to the lower chamber for further incubation.

For calculation of FITC-Dextran concentration, the fluorescence was compared to a calibration curve.

### In Vitro Zonula Occludens-1(ZO-1) Immunostaining.

Retinal pigment epithelium cells (ARPE-19) were cultured on the 4 wells microscope slide Permanox™ Plastic Chamber Slide System (Thermo Scientific, USA, catalog #177437) for 30 days. The cells were washed with starvation medium, incubated with starvation medium containing 0.0, 1.0, 0.1, or 10.0 μg/ml APC for up to 4 hrs, then fixed in 4% paraformaldehyde in phosphate buffer saline (PBS) and, finally, permeabilized with 0.2% of the nonionic surfactant Triton™ X-100 in PBS for 10 minutes. Prior to antigen application, antigen retrieval was performed using 10 mM citric acid pH 6.0 for 10 minutes at 95° C. Demonstration of the antigen was significantly improved by pretreatment with the antigen retrieval reagents that broke the protein cross-links formed by paraformaldehyde fixation, thereby uncovering hidden antigenic sites. The samples were incubated with rabbit anti-ZO-1 antibody (Invitrogen, catalog #40-2300, 1:100) overnight at 4° C. in humidified chamber, followed by incubation with the secondary antibody Alexa Fluor® 568 donkey anti-rabbit antibody (Invitrogen, catalog #A10042, 1100) for 1 hour at room temperature, and 5 min incubation with NucBlue® Fixed Cell ReadyProbe® reagent (Molecular Probesrm, USA; catalog #R37606) diluted in PBS. Images of representative slides were captured digitally using a standard microscope and camera settings. For 3 dimensional (3D) confocal images, 0.5 μm distance Z stack images of cells were captured (Leica TCS SP8). Three-dimensional representation of images was done with the scientific software module Imaris x 64 7.1.1 (Oxford Instruments, UK).

### In Vivo Laser-Induced CNV Animal Models

Choroidal laser photocoagulation is a commonly used method for the induction of choroidal neo vascularization (CNV). Eight weeks old male C57BL/6J mice weighing 19 to 25 grams were anesthetized with intraperitoneal (IP) injection of 100 mg/kg ketamine and 10 mg/kg xylazine, and the pupils were dilated with topical administration of 0.8% tropicamide eye drops. CNV was induced based on the procedure described in Weinberger et al., 2017 (Weinberger et al., Curr Eye Res. 2017, 42:1545-1551) or Livnat at al. (Livnat et al., Exp Eye Res. 2019, 186:107695). Briefly, three laser beams were applied to the right eye using an indirect diode laser ophthalmoscope (IRIS Medical™ Oculight® SLx System, Iridex, Mountain View, Calif., USA) with the treatment beam set at 810 nm. Light rays were focused onto the retinal surface by condensing lenses of 90 diopters (D) (Volk® Optical, Mentor, Ohio, USA). CNV was induced using laser power of 350 mW for 100 msec on the right eye at the 3, 6, and 9 o'clock positions of the posterior pole, at a distance of 1-2 optic disc diameters (DD) surrounding the optic nerve. Formation of white bubble was confirmed by the operator. Immediately following CNV induction, or 1 week after CNV induction, the eyes were intravitreally injected under an operating microscope (Zeiss Opmi 6S Microscope; Carl Zeiss Microscopy GmbH, Germany) with at least one of the following: 1 μl (per mice) of APC (0-5 μg/μl); 1 μl (per mice) of 3K3A-APC (0.1-0.82 μg/μl); saline (as control); or bevacizumab (Avastin®) (25 μg/1 μl) (Genentech, Inc., South San Francisco, Calif., USA and Roche, Basel, Switzerland), using a microsyringe (33-gauge; Hamilton®) placed intravitreally in the retro lateral space of the eye.

For Tie2 blocking experiments, 25 mg/kg Tie2 kinase inhibitor (Calbiochem, catalog #612085, UK) was injected IP prior to CNV induction using the procedure of Minhas et al., 2017 (Minhas et al., Cell Mol Life Sci. 2017, 74:1895-1906).

### Dextran Perfusion and Flattening of Choroid-Retina

For dextran perfusion, mice were anesthetized as described above, and 0.1 ml fluorescein isothiocyanate dextran conjugate (FITC-dextran; MW 500 k, Sigma Aldrich, Rehovot, Israel) diluted in saline to a concentration of 25 mg/ml, was injected to the left ventricle of the mice. Five minutes later, the mice were sacrificed, eyes were enucleated, and a flat specimen of RPE-choroid was separated from the eyecup of each mouse and flattened on glass slides. Three to 4 radial incisions were used to flatten the RPE-choroid. Flattened specimen was washed in PBS and fixed in 4% para formaldehyde (PFA) for 10 minutes. The slides were covered with anti-fade reagent (ProLong™ Gold Antifade Mountant; Invitrogen (Thermo Fisher Scientific)) in order to protect the fluorescent dye from fading (photobleaching) during the fluorescence microscopy experiments. The flattened specimens mounted with anti-fade reagent are referred to herein as “flatmount”.

For immunostaining, flattened specimens were further stained with antibodies and then mounted with an anti-fade reagent.

### Vascular Imaging

Five days post laser applications, mice were anesthetized and treated with FITC dextran conjugate and then sacrificed as described above. For vascular imaging, flat specimen of RPE-choroid was separated from the eye cup and flattened on slides. The slides were covered with ProLong™ Gold Antifade Mountant (Invitrogen). Images of 3 dimensional (3D) projections were captured using the Leica TCS SP8 confocal microscope (Leica Biosystems, Germany). The size of scanned area (x-y plane) was designed to contain the entire laser spot area. The specimens were scanned at the depth of 20 μm starting from the RPE down through Bruch's membrane towards the choriocapillaris. Approximately 582×582 μm² were scanned and 1 μm distance Z-stack images were taken under identical conditions. The volume and depth of staining were measured using the Imaris x64 7.1.1 software (Oxford instruments, UK).

### Immunostaining of Flattened Choroid-Retina Specimens.

For staining of flattened choroid-retina with antibodies, slides obtained as described above were washed with PBS and incubated in a PBS-Triton™ X100 0.5% solution at 4° C. overnight. Slides were then blocked for 1 hours at room temperature (RT) in 5% normal donkey serum (NDS) and incubated with rat anti-mouse CD31 (eBioscience®, San Diego, Calif., USA, catalog #14-0311) 1:100 at 4° C. overnight. For anti-VEGF immunostaining, slides were incubated with rabbit anti-mouse VEGF antibody (1:200; Abcam, UK) at 4° C. overnight. All slides were then washed with PBS and incubated at 4° C. overnight with Alexa Fluor® 568 conjugated goat anti-rabbit IgG (1:100; Invitrogen, USA). Optionally, the slides were further incubated for 15 minutes with 10 μg/ml of the nucleic acid dye 4′,6-Diamidino-2-phenylindole dihydrochloride (DAPI) in PBS (Sigma, catalog #9542). Slides were covered with anti-fade reagent (ProLong®™ Gold Antifade Mountant). A specimen incubated with non-immune serum was used as staining control.

Images of 3 dimensional (3D) projections were captured using the Leica TCS SP8 confocal microscope (Leica Biosystems, Germany). One micrometer (1 μm) distance Z stack images of immunostained flattened retinas-choroid were taken under identical conditions.

The volume and depth of staining were measured using the Imaris x64 7.1.1 software (Oxford Instruments, UK). The CNV area was quantified with Image-J software (NIH, Bethesda, Md., USA). Area of lesions was delineated. Analysis of variance was used for statistical analysis using one-way ANOVA test.

### Fluorescein Angiography (FA)

After mice were anesthetized, their pupils were dilated using tropicamide 0.5%, and 0.1 ml 2.5% fluorescein sodium (Novartis, Switzerland) was injected IP. Sequential real time photos were captured during the early phase (the first minute from fluorescein injection) and late phase (every minute between 2 to 5 minutes from fluorescein injection). Color fundus photographs and fluorescein angiography (FA) images were taken using the Optos® California UWF imaging system (Optos Inc., USA).

Two masked retina specialists evaluated the fluorescein angiograms and sorted each laser spot as “Leakage”, hyperfluorescent lesion with blurred margins increasing in size over time; or “Scar”, hyperfluorescent lesion with distinct margins over time. Other FA abnormalities were also indicated.

### Crayosections Histology and Immunofluorescence Staining

On days 1-30 post APC treatment, mice were sacrificed (n=4 mice per group, a total of 32 mice). Eyes were removed, punched with a 30 g needle, and fixed in 4% PFA for 2 hour at RT. Eyes were washed with increasing concentrations of sucrose in PBS and gradually incubated with a final concentration of 30/sucrose overnight at 4° C. Eyes were then embedded in Tissue-Tek® O.C.T.™ Compound (Sakura Finetek, Japan) on dry ice and kept at −80° C. (this compound is a formulation of water soluble glycols and resins, providing a convenient specimen matrix for cryostat sectioning at temperatures of −10° C. and below). Serial sections of 10 μm thickness were cut using a cryostat (Leica Biosystems, Germany). Sequential crayosections of every fifth section of each eye, were stained with anti-VEGF antibodies as follows, blocked with 10% normal donkey serum (NDS) for 1 hour at RT and incubated with rabbit anti-mouse VEGF antibody at 4° C. overnight (1:400, followed by incubation with Alexa Fluor 568 conjugated goat anti-rabbit IgG (1:100). Finally, nuclei were counterstained with DAPI (NucBlue™ Fixed Cell stain, Molecular Probes, USA). Images were captured under the same settings, using a fluorescence microscope (Axio Imager.Z2, Carl Zeiss Microscopy GmbH, Germany). VEGF staining from lesion sites, was scored from 0-3 (light to heavy, respectively) by 2 masked observers for a total of 5 slides per mouse and 20 photos per group. Sequential crayosections with comparing regions were histologically stained by Hematoxylin and Eosin (H&E) staining for viewing cellular and tissue structure detail (ScyTek Laboratories inc., USA). Images were captured using a fluorescence microscope (Axio Imager.Z2).

### Statistical Analysis

Statistical analysis was performed using SPSS version 23 (IBM Corp., Armonk, N.Y., USA). The data were presented as mean standard deviation (SD). The effect of APC on CNV and VEGF volume and depth was evaluated using one-way ANOVA followed by Tukey post hoc test. The effect of APC on mean scoring of VEGF staining at four time points was evaluated by unpaired two-tailed Student's t test (the resulting P values were adjusted for the number of time points using Sidak's correction). Differences were considered statistically significant if the P value was less than 0.05.

### Example 1

### The Effects of APC on Cellular Localization of the Tight Junction Protein Zonula Occludens 1 (ZO-1)

Intercellular junctional complexes include tight junction proteins, such as occludin or claudins. These proteins bind to the cytoplasmic phosphoprotein zona occludens (ZO)-1, which links to the cytoskeleton and thereby provides junctional stability. Disruption of ZO-1 may lead to breakdown of tight junctions and an increase in vascular (endothelial) or epithelial permeability. In view of the involvement of ZO-1 in linking the tight junction complex to the cytoskeleton and in maintaining RPE's barrier integrity, the effect of exposure to APC on the cellular localization of ZO-1 was analyzed. Cellular localization of ZO-1 was studied using immunofluorescence staining with anti-ZO1 antibody as described in Materials and Methods.

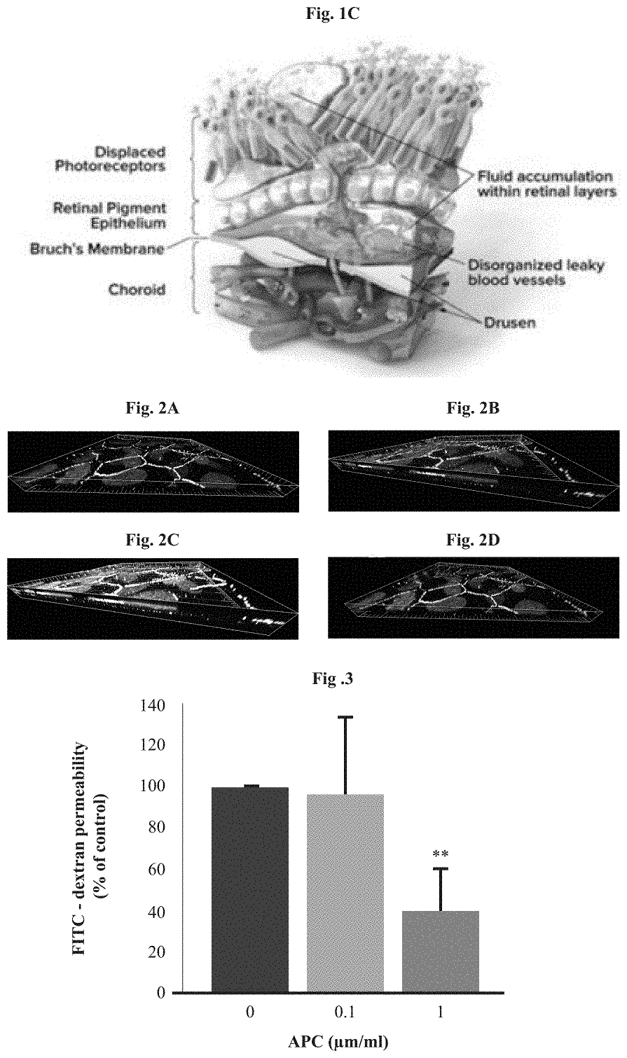

Confocal microscope images of RPE cells stained with rabbit anti-ZO-1 antibody (bright lines) and with NucBlue® Fixed Cell ReadyProbe® reagent, (nuclear staining) with or without APC (0-10 μg/ml) are shown in FIGS. 2A-2D. It is clearly seen that 1 μg/ml APC induced up regulation, translocation and accumulation of ZO-1 protein in the ARPE19 cell membrane. These results suggest that ZO-1 accumulation in the peripheral membrane of epithelial cells may inhibit pathological permeability and angiogenesis through physical barrier formed by tight junction structure.

### Example 2

### Effects of APC on Permeability of RPE Cells In Vitro

To address the question whether translocation of ZO-1 is accompanied by decrease in RPE permeability, cell permeability was evaluated based on spectrophotometric monitoring of the transport of labeled dextran across a cell layer in the absence or presence of APC. In vitro permeability of ARPE-19 cells was assayed according to the protocol described above in Material and Methods.

The results of 4 repeated experiments are summarized in FIG. 3. As shown, exposure of RPE cells to 1 μg/ml APC dramatically reduced leak of FITC-dextran through the RPE monolayer, clearly showing that APC stabilized the RPE barrier.

### Example 3

### Quantitation of CNV Area Following Laser Photocoagulation

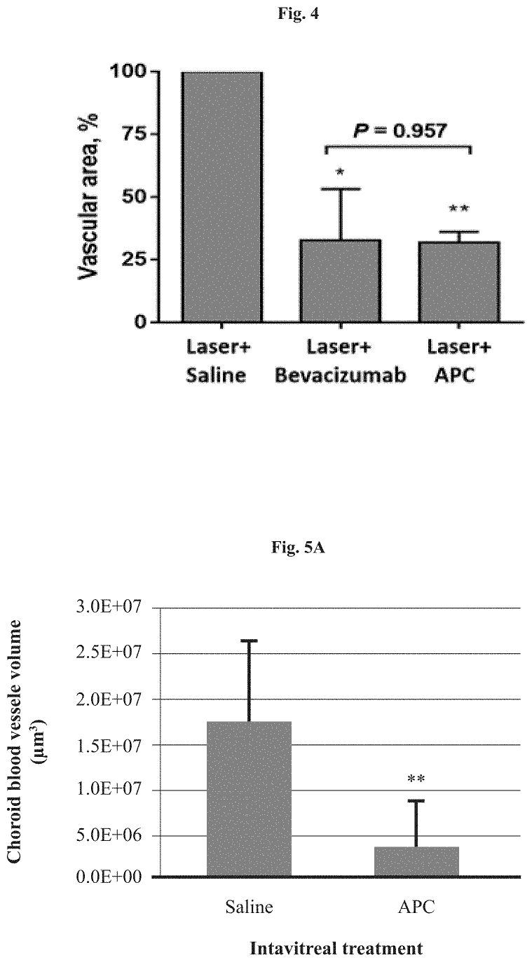

In two repeated experiments, CNV was induced in mice by indirect diode laser photocoagulation as described above in Materials and Methods. Mice were randomly divided into 3 groups (5 mice in each group), and immediately following injury were injected intravitreally with 1 μl saline, 1 μg/μl APC or 25 μg/μl bevacizumab. CNV area was measured 7 days post laser induction in choroidal flatmounts using CD31 immunostaining (anti-CD31 antibody immunofluorescence staining) as described in Materials and Methods. Cluster of differentiation 31 (CD31, also known as platelet endothelial cell adhesion molecule (PECAM-1)), is a protein that makes up a large portion of endothelial cell intercellular junctions. In immunohistochemistry, CD31 is used primarily to demonstrate the presence of endothelial cells and serve as hallmark of CNV. CNV area was evaluated by imageJ photo processing software. Quantification of CNV area (μm²) is presented in FIG. 4. Data are expressed as percent of control (laser application with saline treatment was considered 100%).

As seen in FIG. 4, APC treatment dramatically reduced CNV area. The APC effect was comparable to the effect of bevacizumab, the current treatment of choice for CNV.

### Example 4

### Effect of APC Treatment on CNV Volume and Depth (3-Dimensional Analysis)

Choroidal neo vascularization was induced by indirect diode laser photocoagulation on male C57BL/6J mice as described above in Materials and Methods. Immediately following injury, mice were injected intravitreally with either 1 μl APC at 1 μg/animal or 1 μl saline. Five days after laser applications, mice were anesthetized, and 0.15 ml fluorescein isothiocyanate dextran conjugate (FITC-dextran), diluted in saline to a concentration of 25 mg/ml, was injected into the left ventricle of each rat's heart. Five minutes later, the mice were sacrificed, and their eyes were enucleated. The choroid-RPE and the retina were separated, flattened on slides and mounted with anti-fading reagent as described in Materials and Methods. Images were acquired using a confocal microscope. Quantification of CNV was performed on 3D-reconstructed images using 3D image analysis software.

The bar graphs presented in FIGS. 5A and 5B are the quantification of CNV volume (FIG. 5A) and depth (FIG. 5B) in the choroid-RPE sections. It is clearly demonstrated that APC treatment dramatically reduced CNV volume and depth.

### Example 5

### Effect of a PC on Penetration of Newly Formed Blood Vassals from Choroid into the Retina (3D Analysis)

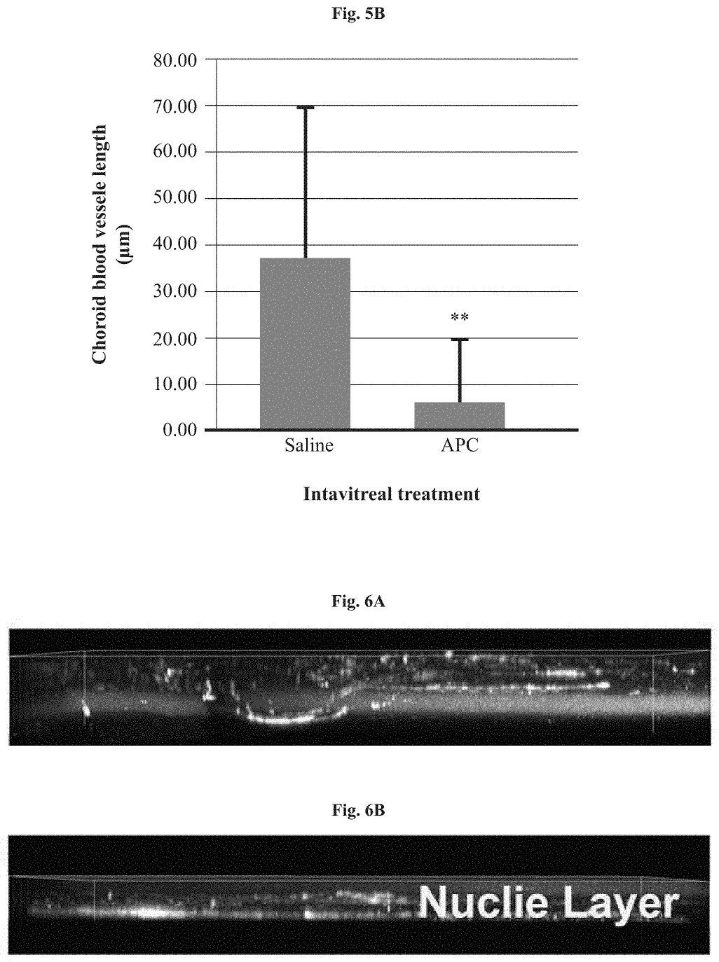

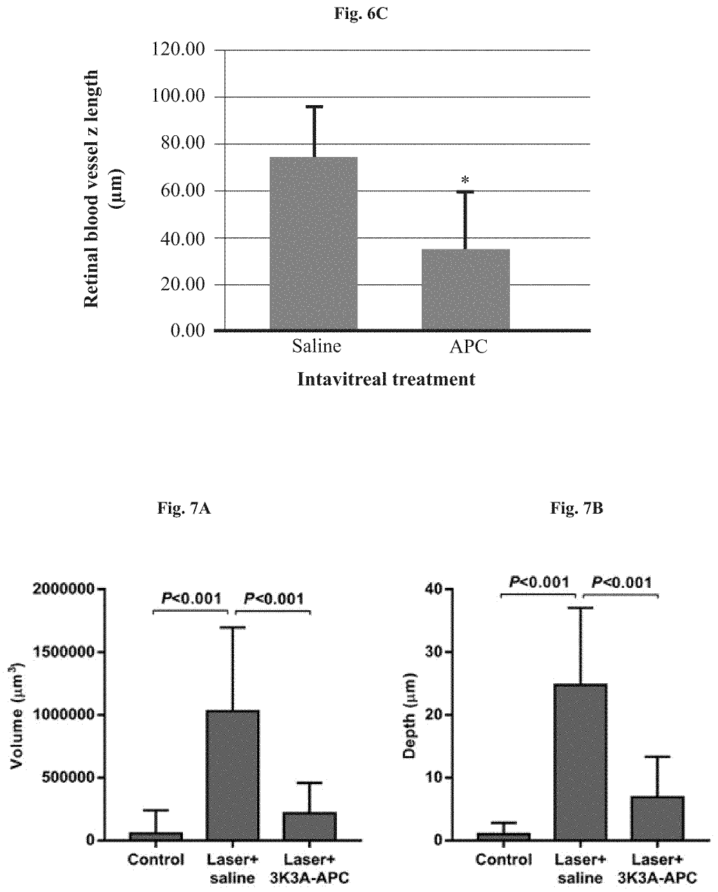

Choroidal neo vascularization was induced in male C57BL/6J mice. Immediately following injury, mice were injected intravitreally with either 1 μl APC at 1 μg/animal or 1 μl saline, and dextran perfusion and flatmounts of the retina were obtained as described above. One micrometer (1 μm) distanced Z stack images of flattened retinas were taken under standard conditions. A summary of Z section measurements of the entire retina 5 days after photocoagulation and following perfusion with dextran is presented in FIGS. 6A-6C.

Representative images of entire retinas of two mice shown in FIGS. 6A-6B demonstrate the appetence of blood vessels at the deeper section of the retina induced by the laser (FIG. 6A, a mouse treated with saline), and inhibition of blood vessels penetration by APC (FIG. 6B, a mouse treated with 1 μl APC).

Quantification of blood vessels area measurements in the retinal sections is shown in FIG. 6C. As clearly seen, APC significantly reduced the penetration of blood vassals from the choroid to the retina.

### Example 6

### Effects of 3K3A-APC on Growth and Invasion of Pre-Existing Blood Vessels from the Choroid into the RPE Layer

Since retinal hemorrhages can cause severe vision impairment, particularly when associated with CNV, the ability of the APC variant 3K3A-APC, which has reduced anticoagulant activity, to retain APCs' activity and reduce CNV growth and penetration toward the sensory retina was tested. CNV was induced in male C57BL/6J mice by laser photocoagulation and verified 6 days later by fluorescein angiography (FA) as described in Materials and Methods. CNV presenting mice were injected intravitreally with 1 μg/μl 3K3A-APC or with saline (7-9 mice per group). Mice without laser application were injected with saline and served as a control. Seven days later, FITC-dextran was perfused from the heart and stained the retina's blood vessels. Choroidal flatmounts were then isolated, and 3 dimensional confocal images were scanned to evaluate the effects of 3K3A-APC on the depth and volume of blood vessels sprouting from the choroid towards the RPE layer. Quantification of the depth (μm), and volume (μm³) of the sprouting vessels were measured using Imaris. The data are presented in FIGS. 7A-7B as mean SD and analyzed using one-way ANOVA followed by Tukey post hoc test.

As seen in the figures, CNV volume was minimal at baseline, without laser application (control) (FIG. 7A). Laser applications have led to an increase in CNV volume, while 3K3A-APC treatment reduced CNV volume (P<0.001). Laser applications have led to an increase in blood vessel depth penetration from 1.1±1.8 to 24.8±12.3 μm (P<0.001) while 3K3A-APC treatment reduced vascular invasion depth to 6.9±6.4 μm (P<0.001) (FIG. 7B).

### Example 7

### The Effect of 3K3A-APC on Retinal Leakage from CNV

Choroidal neo vascularization was induced in male mice as described above. Four after laser application, CNV presence was confirmed by fluorescein angiography (FA) as described in Materials and Methods, and mice (3 laser applications/mouse were injected intravitreally with 1 μg/μl 3K3A-APC (concentration was determined based on dose-dependent analysis previously performed) or saline (7 mice per group). Three days later (7 days after laser application) leakage was measured using FA, as follows: mice were anesthetized and sequential real-time FA images were captured during early phase (i.e., during the first minute from fluorescein injection) and late phase (every minute between 2 to 5 minutes following fluorescein injection). Two masked retina specialists evaluated the fluorescein angiograms and sorted each laser spot as “Leakage”, namely, hyperfluorescent lesion with blurred margins increasing in size overtime or “Scar”, hyperfluorescent lesion with distinct margins overtime.

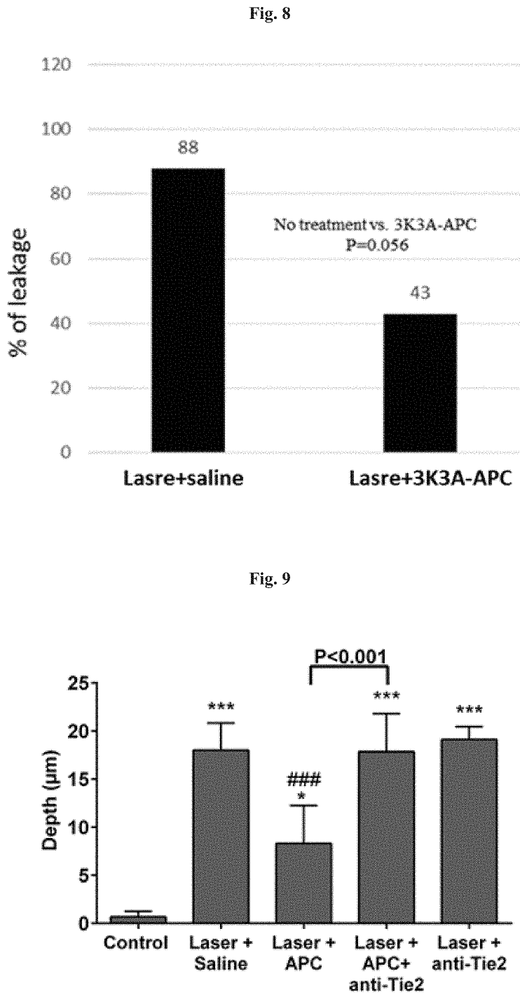

Fluorescein angiography revealed that most of the saline-treated eyes demonstrated leaky CNV characterize by blurred leaking margins increasing in size over time. However, in the 3K3A-APC-treated eyes, the lesion margins remained stable and distinct over time. Quantitative assessment of leaking lesions is presented in FIG. 8.

As seen in FIG. 8, pathologically significant leakage developed in 88% of saline-injected mice, but only in 43% of 3K3A-APC-treated mice. Analysis showed that 3K3A-APC treatment increased the odds of non-leaking lesion status by a factor of 9.333 (with P value of 0.073).

3K3A-APC's ability to reduce leakage and area of pre-existing blood vessels is of great clinical importance since most of the patients are diagnosed with CNV when leaky pathological blood vessels are already present in the retina. The results of this study suggest that injection of 3K3A-APC to patients diagnosed with CNV and retinal leakage may potentially reduce the leakage and growth of pre-existing blood vessels, while simultaneously preventing the formation of new pathological blood vessels.

Data presented herein provide the first proof of concept that 3K3A-APC can be used for modulating pathological CNV. The availability of APC mutants, enabling differentiation between the anticoagulant and cytoprotective activities of this molecule, will deepen our understanding of the biochemical mechanism underlying the potential protective effects of APC against CNV.

### Example 8

### Tie2-Mediated APC Activity in the Retina

Tie2 is a transmembrane endothelial tyrosine kinase receptor is known to play an important role in the protection of endothelial and epithelial barrier functions. APC has been shown by Minhas et al., 2017 (Cell. Mol. Life Sci. https://doi.org/10.1007/s00018-016-2440-6) to be a direct agonist of Tie2 and act as a novel ligand for Tie2 without requiring endothelial protein C receptor (EPCR) or protease-activated receptor-1 (PAR-1). Binding of APC to Tie2 induces rapid activation of the receptor and initiates downstream signaling pathways.

Studies published after the filing date of the parent U.S. patent application Ser. No. 15/780,294 of the present inventors, highlight a novel mechanism by which APC binds directly to Tie2 to enhance endothelial barrier integrity, and strengthen results presented in this patent application, showing APC's protective effects in vascular leakage-related pathologies. In-vivo and in-vitro experiments performed by the present inventors have shown that the protective effects of APC are mediated, at least in part, via the Tie2 receptor.

To examine whether Tie2 is involved in APC-mediated inhibition of pathological choroidal blood vessel growth, Tie2 activity was blocked using a Tie2 kinase inhibitor as previously described (Minhas et al., 2017, supra), and the effects of APC on the depth of blood vessel invasion from the choroid into the RPE layer was studied. Mice were injected intraperitoneally with Tie2 kinase inhibitor prior to CNV induction by laser photocoagulation. Five groups of mice (5 mice per group) were tested: (i) mice that were not treated with Tie2 kinase inhibitor, subjected to laser application immediately followed by intravitreal saline injection; (ii) mice that were not treated with Tie2 kinase inhibitor, subjected to laser application immediately followed by intravitreal APC injection; (iii) mice treated with Tie2 kinase inhibitor, subjected to laser application immediately followed by intravitreal saline injection; (iv) mice treated with Tie2 kinase inhibitor, subjected to laser application immediately followed by intravitreal APC injection: and (v) control group, mice which did not receive any treatment. Seven days post laser application, blood vessels were stained using FITC-dextran perfusion and depth of blood vessels (μm) in choroidal flatmount was analyzed as described above. Quantification of the depth of blood vessels was performed using Imaris. The data are presented in FIG. 9 as mean SD. Data were analyzed using one-way ANOVA followed by Sidak post hoc test.

As seen in FIG. 9, APC reduced the vascular invasion depth induced by laser from 18.0±2.8 μm to 8.3±3.9 μm (P=0.002), while Tie2 blockade has almost completely abolished APC activity and the vascular invasion depth measured was 17.8±4.0 μm (P<0.001 vs. Laser+APC).

### Example 9

### Flatmount Specimens' Evaluation of the Effects of APC on CNV and VEGF Reduction

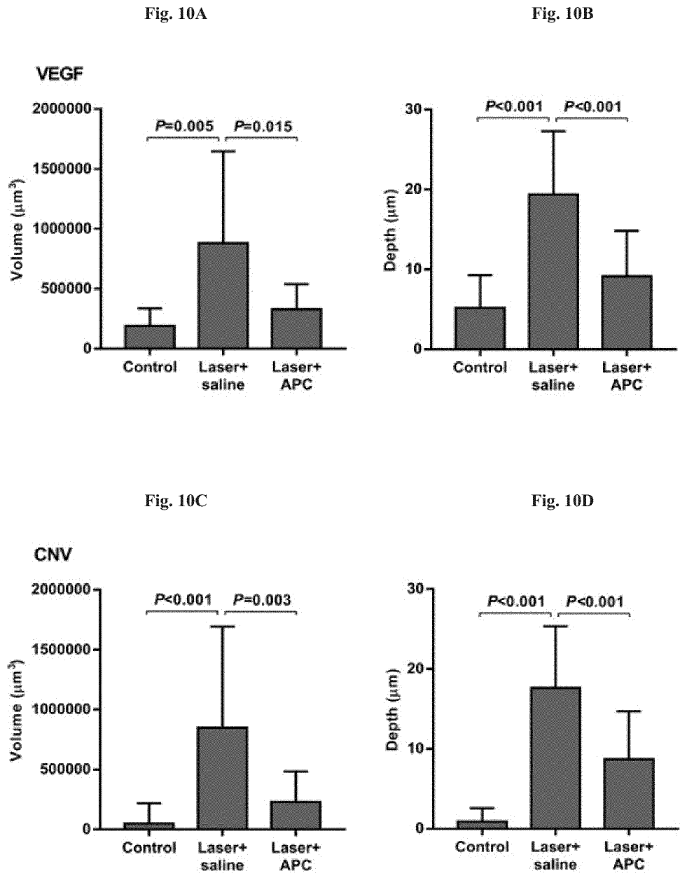

Mice were induced to develop CNV by laser photocoagulation as described above, and 5-7 days post laser application, 7-9 mice per group were injected intravitreally with 1 μg/μl APC or with saline. Mice without laser application were injected with saline and served as a control. Mice were anesthetized 3 days post-APC treatment, and the expression of VEGF was examined, while it is highly expressed. The choroid-RPE specimens (flatmounts) were scanned from the RPE towards the choriocapillaris and VEGF and CNV volume and depth were measured. The results are shown in FIGS. 10A-10D.

A significant reduction in the mean volume of VEGF was measured in the APC-treated mice compared to untreated mice (328457±211489 to 881200±765650 μm³ (P=0.015), respectively). Moreover, the VEGF staining that was demonstrated throughout the entire choroid-Bruchs membrane-RPE depth in the saline-treated eyes (19.4±7.9 μm) was restricted to the RPE region (9.1±5.7 μm depth (P<0.001) in the APC treated eyes (FIGS. 10A-10B). Along with VEGF reduction, APC treatment induced a statistically significant reduction in CNV volume (about 75% reduction), and 50% reduction in the depth of CNV invasion (P<0.001) (FIGS. 10C-10D).

### Example 10

### Time-Dependent Effects of APC on VEGF Levels at CNV Lesion Sites

The present inventors have previously shown that CNV growth was suppressed even 2 weeks following only a single dose injection of APC (Livnat et al., Exp Eye Res. 2019, 186:107695). Now, VEGF levels in cryosections were evaluated 1, 3, 14 and 30 days post-APC (1 μg/μl) treatment as described in Materials and Methods.

A representative image of laser lesion sites taken 3 days post laser photocoagulation is shown in FIGS. 11A-11B. These figures demonstrate that VEGF (red) staining was less prominent at the lesion site of APC treated eyes in comparison to control. FIG. 11C summarizes the mean scoring of VEGF staining 1-30 days post laser application. As expected, VEGF levels increased dramatically after laser application, with a peak at 3 days post laser. A single intravitreal injection of APC induced a statistically significant longitudinal reduction in VEGF levels in the lesion sites 3 days after treatment (P=0.08) and even 14 days post-treatment (P=0.05). Thirty days post-treatment, VEGF levels decreased with no difference between treated and untreated eyes.

### Example 11

### The Effect of 3K3A-A PC on VEGF Levels at CNV Lesion Sites

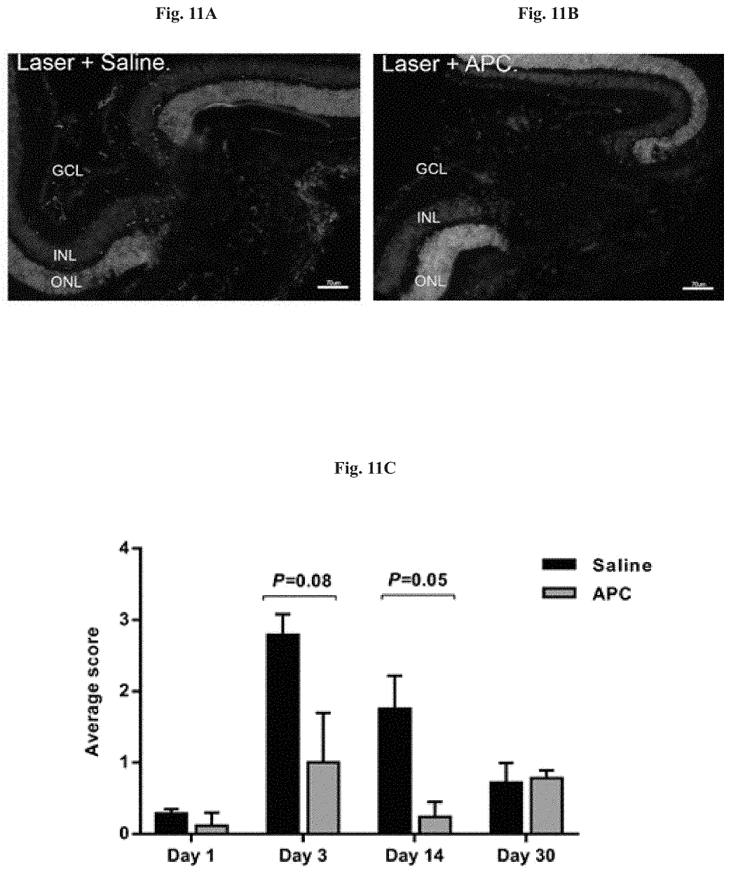

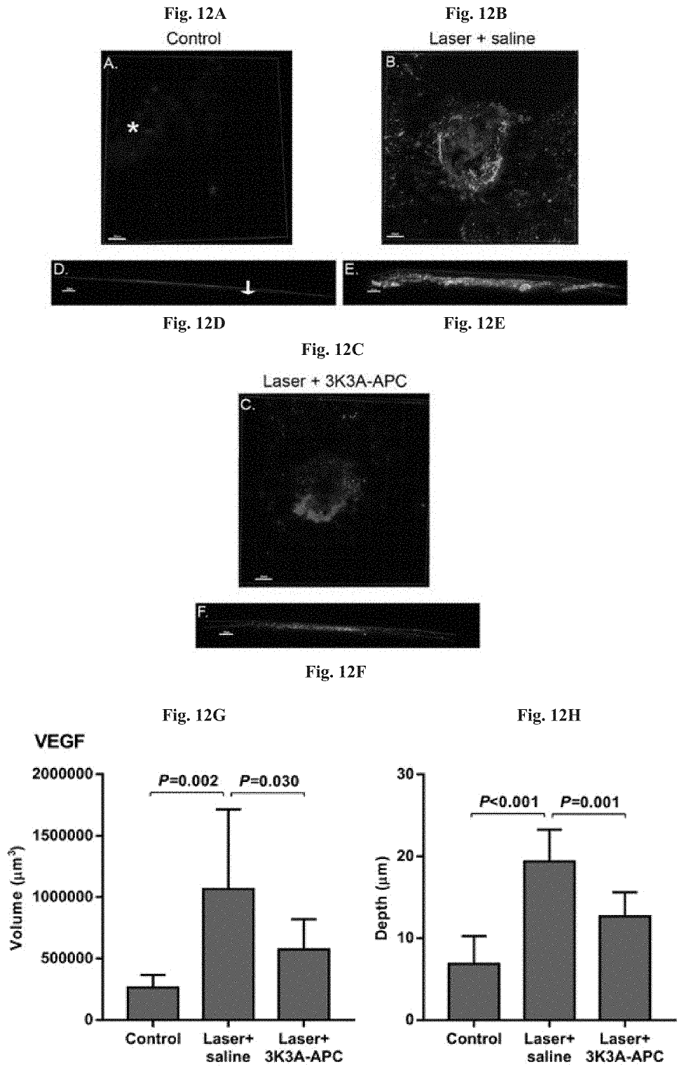

The ability of 3K3A-APC to reduce VEGF levels in CNV was tested. Mice (7-9 mice per group) were subjected to laser photocoagulation and, after CNV was verified, were treated with 1 μg/μl 3K3A-APC or saline. Mice not treated with laser application were injected with saline and served as a control. Three days later, blood vessels were stained using FITC-dextran perfusion, shortly after which mice were sacrificed, choroid-RPE specimens were taken, stained for VEGF (as described in Materials and Methods) and scanned from the RPE layer towards the choriocapillaris. Images of laser-induced lesions immunofluorescently stained for VEGF and quantitative assessment of VEGF levels in the lesions are presented in FIGS. 12A-12H.

An upper view of color images of 3 representing mice of each test group, performed 3 days post-treatment, is shown in FIGS. 12A-12C, and corresponding Z-plane images are shown in FIGS. 12D-12F. In eyes not exposed to laser applications (control eyes; FIG. 12A), only slight staining of VEGF is detected, with a typical hexagonal honeycomb pattern (pointed by the asterisk) corresponding to the edge of the RPE, without vascular staining. Z-axis view shows that VEGF is restricted to the RPE edge, and no CNV staining is detected across the entire depth of the scanned cube (FIG. 12D). In eyes with CNV treated with saline, VEGF and vascular staining are demonstrated throughout the entire depth of choroid-Bruch's membrane-RPE with a strong classic staining appearance of the CNV lesion in the upper side of the RPE (FIGS. 12B, 12E). In eyes treated with 3K3A-APC after laser application, the upper view indicates a dramatic reduction in blood vessels reaching the RPE layer with a significant VEGF staining reduction (FIGS. 12C, 12F). Quantitative assessment of VEGF volume and depth in the entire specimens is shown in FIGS. 12G-12H. The total volume of VEGF in control eyes was 264000±104170 μm³, increased upon laser treatment to 1066286±648214 μm3 (P=0.002) and reduced by 3K3A-APC treatment to 574583±245810 μm³ (P=0.030).

The APC mutant 3K3A-APC maintained the APC activity, and CNV treatment therewith resulted in a reduced vascular invasion depth from 19.4±3.9 μm in laser-treated eyes to 12.7±2.9 μm in the 3K3A-APC treated eyes (P <0.001).

It is evident that many alternatives, modifications and variations of embodiments described herein will be apparent to those skilled in the art. Accordingly, all alternatives, modifications and variations that fall within the spirit and broad scope of the appended claims are encompassed herein.

All publications, patents and patent applications mentioned in this specification are herein incorporated in their entirety by reference into the specification. However, citation or identification of any reference in this application shall not be construed as an admission that such reference is available as prior art to the present disclosure.

What is claimed is:

1. A method for therapy of at least one of choroidal neovascularization (CNV) or retinal leakage associated with CNV in a subject, the method comprising administering to the eye of the subject a therapeutically effective amount of a variant of activated protein C (APC) or a functional partial sequence thereof, thereby providing therapy to the subject.

2. The method of claim 1, wherein the therapy is treatment of retinal leakage and CNV associated with an ocular disease, disorder or condition.

3. The method of claim 1, wherein the ocular disease, disorder or condition is characterized by being at least one of: caused directly by CVN, featuring development of CNV as a secondary stage or a complication thereof, or featuring CNV as a synchronous or asynchronous sequela thereof.

4. The method of claim 3, wherein the ocular disease, disease, disorder or condition is
an ocular disease that is at least one of: age-related macular degeneration (AMD) associated with choroidal neovascularization, pathologic myopia, pseudoxanthoma elasticum with angioid streaks, noninfectious uveitis, infectious uveitis, inflammatory diseases of the optic nerve selected from the group consisting of optic neurtis, papilledema, anterior and ischemic optic neuropath (AION), Behçet's disease or retinopathy;
an ocular disorder that is caused by at least one of: chronic inflammation, oxidative damage, drusen biogenesis, lipofuscin accumulation, abnormalities of Bruch's membrane, vascular changes in the eye that impede regulation of blood pressure and flow and create conditions of ischemia, physiologic aging, genetic factors and environmental factors; or
an ocular condition that is an accidental, occasional incidence in which choroidal neovascularization and retinal leakage develop following a traumatic injury of the retina, or complications during an ophthalmic medical procedure.

5. The method of claim 4, wherein the ocular disease is neovascular age-related macular degeneration (nAMD).

6. The method of claim 1, wherein the functional partial sequence comprises up to 95%, up to 90%, up to 85%, up to 80%, or up to 75% of the amino acid sequence of the APC variant, and it maintains the variant or near variant APC functionality.

7. The method of claim 1, wherein the amino acid sequence of the APC variant is at least 70%, at least 80%, at least 85%, at least 90%, at least 95%, or at least 98%, identical to the amino acid sequence of wild type APC.

8. The method of claim 1, wherein the APC variant is one or more of: 3K3A-APC, RR229/230AA-APC, 5A-APC, APC-2Cys, K193E-APC, E149A-APC, a wild type APC in which residue 158 (Asp) is substituted with a non-acidic amino acid, or residue 154 (His) is substituted with an amino acid residue selected from the group consisting of Lys, Arg or Leu.

9. The method of claim 8, wherein the APC variant is 3K3A-APC.

10. The method of claim 1, wherein the therapy is a combined therapy further comprising administration of one or more active agents selected from the group consisting of an anti-angiogenesis, anti-inflammatory, anti-bacterial, immunosuppressive, anti-PDGF, anti-fungal and anti-viral agent.

11. The method of claim 10, wherein the anti-angiogenesis agent is anti-VEGF agent or platelet derived growth factor (PDGF) inhibitor, and the immunosuppressive agent is a steroid.

12. An ophthalmic composition comprising a variant of activated protein C (APC) or a functional partial sequence thereof, and a carrier acceptable for ophthalmic application.

13. The ophthalmic composition of claim 12, wherein the functional partial sequence comprises up to 95%, up to 90%, up to 85%, up to 80%, or up to 75% of the amino acid sequence of the APC variant, and it maintains the variant or near variant APC functionality.

14. The ophthalmic composition of claim 12, wherein the amino acid sequence of the APC variant is at least 70%, at least 80%, at least 85%, at least 90%, at least 95%, or at least 98%, identical to the amino acid sequence of wild type APC.

15. The ophthalmic composition of claim 12, wherein the APC variant is one or more of: 3K3A-APC, RR229/230AA-APC, 5A-APC, APC-2Cys, K193E-APC, E149A-APC, a wild type APC in which residue 158 (Asp) is substituted with a non-acidic amino acid, or residue 154 (His) is substituted with an amino acid residue selected from the group consisting of Lys, Arg or Leu.

16. The ophthalmic composition of claim 15, wherein the APC variant is 3K3A-APC.
